(19) United States
(12) Patent Application Publication
(10) Pub. No.: US 2017/0119666 A1
(43) Pub. Date: May 4, 2017

(54) STABLE LIPOSOMAL FORMULATIONS OF ALPHA 2 ADRENERGIC AGONISTS FOR OCULAR DELIVERY

(71) Applicant: Peregrine Ophthalmic PTE LTD., Singapore (SG)

(72) Inventors: Ting-Bin Yu, Irvine, CA (US); Zhiwei Shi, Irvine, CA (US); Shih-Horng Su, Irvine, CA (US); Tina Howden, Singapore (SG)

(21) Appl. No.: 14/926,622

(22) Filed: Oct. 29, 2015

(51) Int. Cl. A61K 9/127; A61K 31/498; A61K 9/00; A61K 47/24

(52) U.S. Cl. CPC A61K 9/1271; A61K 47/24; A61K 31/498; A61K 9/0048

(57) ABSTRACT

A stable liposomal formulation for ocular delivery of an alpha 2 adrenergic agonist. The formulation contains a liposome that includes a lipid bilayer including a phosphatidylcholine. An alpha 2 adrenergic agonist is encapsulated in the liposome. Also provided is a method for treating an ocular disorder by administering to the eye the stable liposomal formulation

### BACKGROUND

Elevated intraocular pressure (TOP), also known as ocular hypertension, is a serious medical condition that plays a major role in the pathogenesis of optic nerve damage and glaucomatous visual field loss. Elevated IOP, attributable to an imbalance between inflow and outflow of aqueous humor in the eye, can be effectively reduced by decreasing aqueous humor production by the ciliary body. In the ciliary body, activation of adenylate cyclase leads to an increase in the secondary messenger cAMP, resulting in increased production of aqueous humor. Alpha 2 adrenergic agonists, which inhibit the activity of adenylate cyclase, can potently decrease aqueous humor production.

The alpha 2 adrenergic agonist brimonidine, also known as ALPHAGAN®, is typically prescribed as an eye drop for the purpose of lowering IOP in patients with ocular hypertension and open-angle glaucoma. Typically, as a result of poor absorption of the brimonidine, the eye drops must be administered three times per day at 8 hour intervals. This dosing regimen often leads to patient non-compliance. Additionally, the high brimonidine concentration required in eye drops can have undesirable side effects such as allergic conjunctivitis, conjunctival folliculosis, conjunctival hyperemia, eye pruritus due to topical administration, a burning sensation, oral dryness, and visual disturbances.

Attempts to overcome these problems have focused on non-ionic surfactant vesicles containing sorbitan monostearates as an ocular delivery system for brimonidine. See, e.g., Maiti et al., AAPS PharmSciTech, 12:755-763 and Prabhu et al., J. Young Pharm. 2:356-361. However, sorbitan monostearates are non-natural occurring excipients with the potential for undesirable side effects and systemic toxicity.

The need exists for stable alpha 2 adrenergic agonist formulations for ocular administration which are safer and more effective than existing formulations while, at the same time, offering sustained release and superior stability.

### SUMMARY

To meet the need set forth above, a stable liposomal formulation for ocular delivery is disclosed.

The formulation contains a liposome that includes a lipid bilayer containing a phosphatidylcholine, and an alpha 2 adrenergic agonist encapsulated in the liposome. The weight ratio between the alpha 2 adrenergic agonist and the phosphatidylcholine is 1:10 to 1:100, and the liposome has a diameter of less than 2 μm and is free of cholesterol and a non-ionic detergent.

The lipid bilayer of the liposome can also include, in addition to the phosphatidylcholine, a phospholipid conjugated to a polyethylene glycol moiety.

The alpha 2 adrenergic agonist can be brimonidine, apraclonidine, or clonidine.

The stable liposomal formulation for ocular delivery can be in the form of eye drops. In a preferred embodiment, the stable liposomal formulation is in the form of an injectable solution.

Also disclosed is a method for treating an ocular disorder by administering to the eye the stable liposomal formulation described above. The ocular disorder can be, e.g., ocular hypertension or glaucoma. In a particular aspect, the glaucoma is open-angle glaucoma.

To effect treatment, eye drops containing the stable liposomal formulation are applied topically to the eye. Alternatively, the formulation is administered via subconjunctival injection.

Furthermore, the stable liposomal formulation can release the alpha 2 adrenergic agonist encapsulated in the liposome over an extended period of time. For example, the alpha 2 adrenergic agonist can be release from the liposome continuously over a period of up to 3 months after administration of the formulation.

The details of one or more embodiments of the invention are set forth in the drawings and description below. Other features, objects, and advantages of the invention will be apparent from the description, from the drawing, and from the claims. All references cited herein are hereby incorporated by reference in their entirety.

### BRIEF DESCRIPTION OF THE DRAWING

The invention description below refers to the accompanying drawing.

FIG. 1 is a plot of the in vitro release profiles of aqueous brimonidine (AqBM; squares) and liposomal brimonidine (LipoBM; diamonds).

### DETAILED DESCRIPTION

As mentioned above, a stable liposomal formulation for ocular delivery of an alpha 2 adrenergic agonist is provided. The alpha 2 adrenergic agonist is encapsulated in a liposome that includes a lipid bilayer containing a phosphatidylcholine. The phosphatidylcholine can be one or more of egg phosphatidylcholine (EggPC); palmitoyl oleoyl phosphatidylcholine (POPC); 1,2-dimyristoyl-sn-glycero-3-phosphocholine (DMPC); and 1,2-dioleoyl-sn-glycero-3-phosphocholine (DOPC). In a preferred embodiment, the phosphatidylcholine is POPC. The stable liposomal formulation, in an embodiment, contains a plurality of liposomes each having the above-described composition.

In addition to the phosphatidylcholine, the lipid bilayer of the liposomes can further include a phospholipid conjugated to a polyethylene glycol (PEG) moiety. The PEG-conjugated phospholipid can be 1,2-distearoyl-sn-glycero-3-phosphoethanolamine-N-[amino(PEG)](PEG-DSPE); 1,2-dioleoyl-sn-glycero-3-phosphoethanolamine-N-[methoxy(PEG)](PEG-DOPE); 1,2-dipalmitoyl-sn-glycero-3-phosphoethanolamine-N-[methoxy(PEG)](PEG-DPPE); 1,2-dimyristoyl-sn-glycero-3-phosphoethanolamine-N-[methoxy(PEG)](PEG-DMPE); or mixtures of these conjugated phospholipids. Preferably, the PEG-conjugated phospholipid is PEG-DSPE.

The molecular weight of the PEG moiety can be 350 to 3000 g/mol, e.g., 350, 500, 750, 1000, 1250 1500, 1750, 2000, 2250, 2500, 2750, 3000 g/mol. In a preferred embodiment, the molecular weight of the PEG moiety is 2000 g/mol.

Importantly, the lipid bilayer defined above is free of cholesterol and any non-ionic detergents.

As mentioned above, an alpha 2 adrenergic agonist is encapsulated in the liposomes. The alpha 2 adrenergic agonist can be brimonidine, apraclonidine, or clonidine. In a particular aspect, the alpha 2 adrenergic agonist is brimonidine.

The weight ratio between the alpha 2 adrenergic agonist and the phosphatidylcholine in the formulation can be 1:10 to 1:100 (e.g., 1:10, 1:15, 1:20, 1:25, 1:30, 1:35, 1:40, 1:45, 1:50, 1:75, and 1:100). In an embodiment, the weight ratio is 1:10 to 1:30. In a preferred embodiment, the weight ratio is 1:20.

The above-described liposomes have a diameter of less than 2 μm. In an embodiment, the liposomes are at least 50 nm in diameter and less than 1 μm in diameter. For example, the diameter can be 50 nm, 100 nm, 150 nm, 200 nm, 250 nm, 300 nm, 350 nm, 400 nm, 500 nm, or 1 μm. Preferably, the diameter of the liposomes is between 100 nm and 400 nm. In a particular embodiment, the diameter of the liposomes is 150 nm. In a preferred embodiment, the liposomes are large unilamellar vesicles (LUVs) having the just-mentioned diameters.

The liposomes in the stable formulation have a particular uniformity in diameter. More specifically, the polydispersity index (PDI) of the liposomes is in the range of 0.03 to 0.300. Preferably, the PDI is from 0.100 to 0.300.

In a specific stable liposomal formulation, the liposomes are LUVs, the bilayer contains POPC and PEG-DSPE, where the PEG moiety has a molecular weight of 2000 g/mol (PEG2000-DSPE), the alpha 2 adrenergic agonist is brimonidine, the weight ratio between the brimonidine and the POPC is 1:10 to 1:40, and the liposomes have a diameter of 150 nm.

The stable liposomal formulations described above can have an alpha 2 adrenergic agonist content of 1 mg/mL to 100 mg/mL (e.g., 1 mg/mL, 2.5 mg/mL, 5 mg/mL, 7.5 mg/mL, 10 mg/mL, 12.5 mg/mL, 20 mg/mL, 25 mg/mL, 50 mg/mL, 75 mg/mL, and 100 mg/mL). In an embodiment, the stable liposomal formulation has an alpha 2 adrenergic agonist content of 1 mg/mL to 20 mg/mL. In a particular aspect, the alpha 2 adrenergic agonist content is 8 mg/mL.

The alpha 2 adrenergic agonist in the stable liposomal formulations described above is stable for at least 12 weeks when stored at 5° C. as compared to the same drug in an aqueous solution. In this context, stability is defined as a loss of no more than 20% of the starting amount of the drug in the formulation over the incubation period.

The stable liposomal formulation can release the encapsulated alpha 2 adrenergic agonist over an extended period of time. For example, the alpha 2 adrenergic agonist can be release from the liposome after administration of the formulation continuously over a period of up to 3 months, e.g., 7, 14, 21 days and 1, 2, and 3 months.

The stable liposomal formulation for ocular delivery of an alpha 2 adrenergic agonist can be produced by a thin-film hydration method. A thin film is formed after dissolving a phosphatidylcholine and the alpha 2 adrenergic agonist in a solvent and then completely evaporating the solvent. The solvent can be methanol, ethanol, chloroform, or mixtures thereof. The thin film can have a thickness of 0.1 μm to 1500 μm. The thin film is hydrated in phosphate-buffered saline (PBS) to form a solution of multi-lamellar vesicles (MLVs). LUVs are formed from the MLVs by an extrusion process. For example, the MLVs can be extruded through a polycarbonate filter from 3 to 10 times. The pore size of the filter can range from 50 nm to 200 nm.

Preferably, the stable liposomal formulation described above is prepared for ocular delivery. For example, the stable liposomal formulation can be in the form of eye drops. In a preferred embodiment, the stable liposomal formulation is in the form of an injectable solution.

The stable liposomal formulation set out, supra, can be used for treating an ocular disorder. The ocular disorder can be, e.g., ocular hypertension or glaucoma. In a particular aspect, the glaucoma is open-angle glaucoma.

As mentioned above, a method for treating an ocular disorder is provided. The method includes administrating the stable liposomal formulation of an alpha 2 adrenergic agonist to the eye of a subject. For example, eye drops containing the formulation can be applied to the eye. Alternatively, in a preferred method, the formulation is administered via subconjunctival injection.

The stable liposomal formulations that can be used in the method for treating an ocular disorder include an alpha 2 adrenergic agonist encapsulated in a liposome. The alpha 2 adrenergic agonist can be any drug having the ability to act as an alpha 2 adrenergic agonist. In particular aspects, the stable liposomal formulation administered in the method contains brimonidine, apraclonidine, or clonidine. In a particular method, the alpha 2 adrenergic agonist is brimonidine.

The liposomes in the stable liposomal formulation that can be used in the method for treating an ocular disorder have a diameter of less than 2 μm. In an embodiment, the liposomes are at least 50 nm in diameter and less than 1 μm in diameter. For example, the diameter can be 50 nm, 100 nm, 150 nm, 200 nm, 250 nm, 300 nm, 350 nm, 400 nm, 500 nm, or 1 μm. Preferably, the diameter of the liposomes is between 100 nm and 400 nm. In a particular embodiment, the diameter of the liposomes is 150 nm. In a preferred embodiment, the liposomes are LUVs having the just-mentioned diameters.

As mentioned above, the stable liposomal formulation can release the alpha 2 adrenergic agonist in a sustained manner. As such, the method for treating an ocular disorder can include administering the formulation at extended intervals. For example, the alpha 2 adrenergic agonist-containing stable liposomal formulation can be applied to the eye once every week, once every 2 weeks, or once every 1-6 months (e.g., 1, 2, 3, 4, 5, and 6 months).

The frequency of administration can be adjusted depending on the mode of administration. For example, the stable liposomal formulation is administered in the form of eye drops once per week or once every two weeks. The stable liposomal formulation can be administered by subconjunctival injection once every 1-6 months (e.g., once per month, once every 2 months, once every 3 months, once every 4 months, once every 5 months, and once every 6 months).

In a particularly preferred embodiment, the method entails treating an open-angle glaucoma patient by injecting subconjunctivally a stable liposomal formulation of brimonidine, where the liposomes include POPC and PEG2000-DSPE, the weight ratio between the brimonidine and the POPC is 1:20, and the liposomes have a diameter of 150 nm.

Without further elaboration, it is believed that one skilled in the art can, based on the description above, utilize the present invention to its fullest extent. The specific examples below are to be construed as merely illustrative, and not limitative of the remainder of the disclosure in any way whatsoever.

### Example 1: Preparation of Brimonidine-Loaded Liposomes Containing PEG-Ylated Phospholipid LUVs

50 mg of brimonidine tartrate, 1000 mg of POPC, and 100 mg of DSPE-PEG2000 (ammonium salt) were added to 3 mL of ethanol. The resulting solution was stirred until it appeared clear. A nitrogen gas stream was used to completely evaporate the ethanol at room temperature for at least 90 min, to form a transparent thin film. 3 mL of PBS at pH 7.4 was added to the film and stirred for at least 60 min, thereby forming a milky-white suspension of multilamellar vesicles (MLVs). The sizes of the MLVs were reduced by extrusion through a 3-stack of polycarbonate filter membranes (pore size 100 nm) using a bench top extruder (Northern Lipids Inc., Canada). After 4 extrusion passes, large unilamellar vesicles (LUVs) with an average size of ˜150 nm were obtained. The brimonidine-loaded PEGylated liposomal formulation (“LipoBM”) was analyzed for drug content, mean vesicle size, and polydispersity index (PDI). The physical characteristics of the brimonidine-loaded liposomes were determined essentially as described in Venkatraman et al., International Application Publication No. 2012/021107, the content of which is incorporated herein by reference in its entirety. The results are shown in Table 1 below.

TABLE 1
Physical properties of Brimonidine-loaded PEGylated Liposomes

| Properties | Measured Values | |
|---|---|---|
| brimonidine content | 7.92 | mg/mL |
| target POPC content | 300.0 | mg/mL |
| brimonidine to POPC ratio | 1:37.88 | |
| mean vesicle size | 145 | nm |

| | |
|---|---|
| PDI | 0.211 |

### Example 2: Measurement of Brimonidine Encapsulation Efficiency

Brimonidine-loaded liposomes were prepared as described above in EXAMPLE 1. The encapsulation efficiency of brimonidine in the liposomes was evaluated by employing gel-filtration to remove free brimonidine-HCl from the liposomal preparations. The drug to lipid ratio of liposomal formulation samples were determined before and after running them on a PD-10 cross-linked dextran gel (SEPHADEX® G-25) desalting column using the following equation:

$$Drug Encapsulation \% = \frac{\begin{matrix}Final Drug : Lipid Ratio \\ ( samples passed through PD \text{-} 10 column )\end{matrix}}{\begin{matrix}Initial Drug : Lipid Ratio \\ ( samples prior to PD \text{-} 10 separation )\end{matrix}} \times 100 \%$$

The drug encapsulation efficiency of liposomal brimonidine solution was 56.0%.

### Example 3: In-Vitro Drug Release Study

Drug release studies were performed by dialyzing both LipoBM prepared as described above in EXAMPLE 1 and an aqueous brimonidine solution (“AqBM”) against PBS at a pH of 7.4 and measuring by HPLC the amount of brimonidine released.

Briefly, 1 mL of AqBM and 1 ml LipoBM were each loaded into a dialysis tube (molecular weight cut-off of 8-10 kDal) before placing the tube into 40 mL of PBS in a 50 mL tube. The PBS in the tubes was sampled at 1, 4, 7, 24, 48, 120, 144, and 168 hours at which time the brimonidine concentration was measured by HPLC.

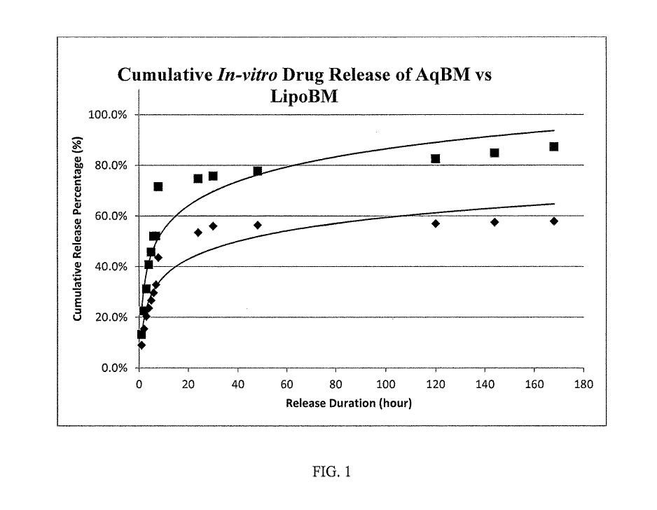

The cumulative drug release percentage vs release time for both AqBM and LipoBM was calculated and plotted. The results, shown in FIG. 1, indicated that ˜80% of the unencapsulated brimonidine (AqBM; squares) diffused through the dialysis tubing within 48 hours while, at the same time, only 56% of the encapsulated brimonidine (LipoBM; diamonds) was released. After 168 hours, almost 90% of unencapsulated brimonidine was released, whereas only 58% of encapsulated brimonidine was released. Undoubtedly, encapsulation of brimonidine in a liposome results in a formulation capable of sustained drug delivery.

### Example 4: Stability of Brimonidine-Loaded Liposomes

The brimonidine content of a LiopBM formulation produced as described above in EXAMPLE 1 was analyzed after storing it at 5° C. for up to 12 weeks. The temperature was monitored and recorded continuously to ensure stable temperature conditions. The results are shown in TABLE 2 below.

TABLE 2
Stability of brimonidine-loaded liposome (LipoBM)

| | weeks of storage | | | | | |
|---|---|---|---|---|---|---|
| | 0 | 1 | 2 | 3 | 4 | 12 |
| drug Content | 7.92 | 7.15 | 7.05 | 7.32 | 7.63 | 7.52 |
| (mg/mL) | | | | | | |
| size (nm) | 145.3 | 145.3 | 143.8 | 150.0 | 150.3 | 148.15 |
| PDI | 0.211 | 0.211 | 0.198 | 0.237 | 0.282 | 0.244 |

No significant changes in drug content, size, and PDI were observed throughout the stability study. It is clear that LipoBM is stable for at least 12 weeks upon storage at 5° C.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

1. A stable liposomal formulation for ocular delivery, the formulation comprising a liposome including a lipid bilayer that contains a phosphatidylcholine, and an alpha 2 adrenergic agonist encapsulated in the liposome, wherein a weight ratio between the alpha 2 adrenergic agonist and the phosphatidylcholine is 1:10 to 1:100, and the liposome has a diameter of less than 2 μm and is free of cholesterol and a non-ionic detergent.

2. The stable liposomal formulation of claim 1, wherein the alpha 2 adrenergic agonist is brimonidine, apraclonidine, or clonidine.

3. The stable liposomal formulation of claim 2, wherein the liposome has a diameter Of 100 nm to 400 nm.

4. The stable liposomal formulation of claim 2, wherein the phosphatidylcholine is egg phosphatidylcholine (EggPC), palmitoyl oleoyl phosphatidylcholine (POPC), 1,2-dimyristoyl-sn-glycero-3-phosphocholine (DMPC), or 1,2-dioleoyl-sn-glycero-3-phosphocholine (DOPC).

5. The stable liposomal formulation of claim 4, wherein the liposome has a diameter of 100 nm to 400 nm.

6. The stable liposomal formulation of claim 4, wherein the lipid bilayer further contains a polyethylene glycol-conjugated phospholipid.

7. The stable liposomal formulation of claim 6, wherein the liposome has a diameter of 100 nm to 400 nm.

8. The stable liposomal formulation of claim 6, wherein the polyethylene glycol-conjugated phospholipid is 1,2-distearoyl-sn-glycero-3-phosphoethanolamine-N-[amino(polyethylene glycol)], 1,2-dioleoyl-sn-glycero-3-phosphoethanolamine-N-[methoxy(polyethylene glycol)], 1,2-dipalmitoyl-sn-glycero-3-phosphoethanolamine-N-[methoxy(polyethylene glycol)], or 1,2-dimyristoyl-sn-glycero-3-phosphoethanolamine-N-[methoxy(polyethylene glycol)].

9. The stable liposomal formulation of claim 8, wherein the liposome has a diameter of 100 nm to 400 nm.

10. The stable liposomal formulation of claim 8, wherein each of the polyethylene glycol moieties has a molecular weight of 350 to 3000 g/mol.

11. The stable liposomal formulation of claim 10, wherein the liposome has a diameter of 100 nm to 400 nm.

12. The stable liposomal formulation of claim 8, wherein the polyethylene glycol-conjugated phospholipid is 1,2-distearoyl-sn-glycero-3-phosphoethanolamine-N-[amino(polyethylene glycol)], the polyethylene glycol has a molecular weight of 2000 g/mol, the phosphatidylcholine is POPC, the alpha 2 adrenergic agonist is brimonidine, the weight ratio between the brimonidine and the POPC is 1:10 to 1:40, and the liposome has a diameter of 150 nm.

13. A method for treating an ocular disorder, comprising administering to an eye of a subject in need thereof the stable liposomal formulation of claim 1.

14. The method of claim 13, wherein the ocular disorder is ocular hypertension or open angle glaucoma.

15. The method of claim 14, wherein the stable liposomal formulation is administered topically or via subconjunctival injection.

16. The method of claim 13, wherein the lipid bilayer further contains a polyethylene glycol-conjugated phospholipid.

17. The method of claim 16, wherein the ocular disorder is ocular hypertension or open angle glaucoma.

18. The method of claim 17, wherein the stable liposomal formulation is administered topically or via subconjunctival injection.

19. The method of claim 16, wherein the polyethylene glycol-conjugated phospholipid is 1,2-distearoyl-sn-glycero-3-phosphoethanolamine-N-[amino(polyethylene glycol)], the polyethylene glycol has a molecular weight of 2000 g/mol, the phosphatidylcholine is POPC, the alpha 2 adrenergic agonist is brimonidine, the weight ratio between the brimonidine and the POPC is 1:10 to 1:40, and the liposome has a diameter of 150 nm.

20. The method of claim 19, wherein the ocular disorder is ocular hypertension or open angle glaucoma.
